(19) United States
(12) Patent Application Publication
(10) Pub. No.: US 2004/0192645 A1
(43) Pub. Date: Sep. 30, 2004

(54) Amyloid plaque as a target for therapeutics that function by blocking or disrupting chitin synthesis or activity

(75) Inventors: Rawle I. Hollingsworth, Haslett, MI (US); Birgit Zipser, East Lansing, MI (US); Linjuan Huang, East Lansing, MI (US)

Correspondence Address: MCLEOD & MOYNE, P.C., 2190 COMMONS PARKWAY, OKEMOS, MI, 48864, US

(73) Assignee: Board of Trustees of Michigan State University, East Lansing, MI

(21) Appl. No.: 10/795,652

(22) Filed: Mar. 8, 2004

Related U.S. Application Data
(60) Provisional application No. 60/453,432, filed on Mar. 10, 2003.

(51) Int. Cl. A61K 31/722

(52) U.S. Cl. 514/55

(57) ABSTRACT

Chitin has been discovered to accumulate in the diseased tissue of mammals, including humans, afflicted with a disease characterized by formation of congo red-staining plaques. Such diseases include Alzheimer's disease, spongiform encepalopathies, type II diabetes, atrial amyloidosis, and the like. A method for detecting the chitin in the mammal is described which is useful for diagnosing disease caused by accumulation of the chitin or amyloid plaques comprising chitin in tissue. Further described is a method for treating a disease in the mammal caused by the accumulation of chitin or amyloid plaques comprising chitin by administering a composition which inhibits formation of the chitin or degrades the chitin.

### CROSS-REFERENCE TO RELATED APPLICATION

This application claims priority to U.S. provisional application Ser. No. 60/453,432, filed Mar. 10, 2003.

Reference to a “Computer Listing Appendix submitted on a Compact Disc”

Not Applicable.

### STATEMENT REGARDING FEDERALLY SPONSORED RESEARCH OR DEVELOPMENT

This invention was made in the course of work supported by a National Institutes of Health Grant No. NS25117. Therefore, the U.S. Government has certain rights in the invention.

### BACKGROUND OF THE INVENTION

(1) Field of the Invention

The present invention relates to a method for detecting chitin and amyloid plaques which comprise chitin in mammals, including humans. The present invention further relates to a method for diagnosing a disease caused by the accumulation of the chitin or amyloid plaques comprising chitin in tissue of mammals, including humans, wherein the disease is characterized by the formation of congo red-staining bodies which are characteristic of amyloid plaques. The chitin in the amyloid plaque makes detection and imaging of the amyloid plaque possible through means for detecting and imaging the chitin in the amyloid plaque. The present invention further relates to a method for preventing disorders or diseases in mammals, including humans, which are characterized by the formation and accumulation of chitin or amyloid plaques which comprise chitin. In particular, the present invention relates to a method for treating a disorder or disease characterized by the accumulation of chitin or amyloid plaques which comprise chitin in a tissue of the mammal or human by administering to the mammal or human a composition comprising one or more molecules, chemicals, or drugs which inhibit formation of the chitin or chitin in the amyloid plaque, which effect a reduction in formation of the chitin or chitin in the amyloid plaque, or which effect a degradation of the chitin or chitin in the amyloid plaque.

(2) Description of Related Art

Chitin is a carbohydrate homopolymer of β(1→4) linked N-acetylglucosamine chitin synthase belonging to a group of enzymes that catalyze the synthesis of polysaccharides and are known generally as glucosyl transferases. These enzymes are well known to occur in prokaryotic and eukaryotic non-mammalian organisms and are responsible for cellulose and hyaluronic acid synthesis, as disclosed in U.S. Pat. No. 6,465,179 to Thireos et al.

There are well known chitin synthesis inhibitors including Streptomyces antibiotics Nikkomycins and Polyoxins (See U.S. Pat. No. 5,330,976). Benzoylphenyl-ureas are known to inhibit chitin synthesis in arthropods.

Thireos et al. also describe chitin synthase DNA sequences from Drosophila melanogaster. The DNA can be used to assay for inhibitors of chitin synthase as disclosed in the patent.

Chitin synthase is not known in humans per se. A substantially similar synthase is hyaluronic acid synthase which is known to also synthesize chitin in vitro when N-acetylglucosamine is present as the substrate. Hyaluronic acid is present in connective tissue. No connection has been made to the synthesis of chitin in humans by hyaluronic acid synthase.

While humans do not appear to have a chitin synthase, humans do produce a chitinase. The human chitinase has been described in U.S. Pat. Nos. 6,200,951, 6,399,571, and 6,372,212 to Gray et al., wherein the human chitinase, DNA encoding the chitinase, and fragments of the chitinase for detecting chitin, binding chitin, and treating fungal infections. These patents provide a detailed background regarding chitinase which enables the present invention.

### Detection of Diseases

Alzheimer's disease is generally diagnosed post-mortem because of the lack of a means for detecting the disease in living humans. While a satisfactory test for detecting Alzheimer's disease has been elusive goal, some progress has been achieved. For example, Skovronsky et al. (Proc. Natl. Acad. Sci. USA 97: 7609-7614 (2000) describe a first towards the development of an in vivo method for detecting amyloid plaques in mouse brains using a radioligand comprising (trans,trans)-1-bromo-2,5-bis-(3-hydroxycarbonyl-4-hydroxy)styrylbenzene. However, the method has not been used to detect amyloid plaques in the brains of living mammals. Frenkel and Solomon (Proc. Natl. Acad. Sci. USA 99: 5675-5679 (2002); U.S. Patent Application No. 20020052311) disclose a method which uses filamentous phage comprising a single chain Fv antibody polypeptide specific for the amyloid protein. However, this method has not been used to detect amyloid plaques in the brains of living animals. Therefore, there remains a need for a method for detecting Alzheimer's disease in a living mammal, particularly humans.

### Objects

It is an object of the present invention to provide a method for the diagnosis of diseases caused by accumulation of chitin alone or in amyloid plaques.

It is further an object of the present invention to provide molecules which inhibit chitin and amyloid plaque formation and which allow the degradation of the accumulated chitin.

These and other objects of the present invention will become increasingly apparent with reference to the following drawings and preferred embodiments.

### SUMMARY OF THE INVENTION

The present invention provides a method for detecting chitin and amyloid plaques which comprise chitin or a chitin core in mammals, including humans. The present invention further provides a method for diagnosing a disease caused by the accumulation of chitin or amyloid plaques comprising chitin or a chitin core in tissue of mammals, including humans, wherein the disease is characterized by the formation of congo red-staining bodies which are characteristic of amyloid plaques. The chitin or chitin core in the amyloid plaque makes detection and imaging of the amyloid plaque possible through means for detecting and imaging the chitin or chitin core in the amyloid plaque. The present invention further provides a method for preventing disorders or diseases in mammals, including humans, which are characterized by the formation and accumulation of chitin or amyloid plaques which comprise chitin or a chitin core. In particular, the present invention provides a method for treating a disorder or disease characterized by the accumulation of chitin or amyloid plaques which comprise chitin or a chitin core in a tissue of the mammal or human by administering to the mammal or human a composition comprising one or more molecules, chemicals, or drugs which inhibit formation of the chitin or chitin in the amyloid plaque, which effect a reduction in formation of the chitin or chitin in the amyloid plaque, or which effect a degradation of the chitin or chitin in the amyloid plaque.

Therefore, in one embodiment, the present invention provides a method for treating a disorder in a mammal, preferably a human, characterized by accumulation of congo red-staining plaques in a tissue of the mammal which comprises administering to the mammal an effective amount of a composition which inhibits polymerization of N-acetylglucosamine or derivative thereof to form chitin or conjugate thereof to treat the disorder.

In a further embodiment of the method, the disorder is selected from the group consisting of spongiform encepalopathies, Alzheimer's disease, hemodialysis-related amyloidosis, primary systemic amyloidosis, secondary systemic amyloidosis, familial amyloid polyneuropathy I, familial amyloid polyneuropathy III, cerebral amyloid angiopathy, Finnish hereditary systemic amyloidosis, type II diabetes, injection-localized amyloidosis, medullary thyroid carcinoma, atrial amyloidosis, non-neuropathic systemic amylodosis, and hereditary renal amyloidosis.

In a further embodiment of the method, the chitin or conjugate thereof is in the brain or in the circulatory system. In a further embodiment, the chitin or conjugate thereof is in the plaques in the mammal.

In a further embodiment of the method, the disorder is Alzheimer's disease or diabetes and the chitin or conjugate thereof is in the plaques in the brain or the disorder is atherosclerosis and the chitin or conjugate thereof is in the plaques in the blood vessel.

In a further embodiment of the method, the composition comprises an inhibitor of an enzyme which produces the chitin or conjugate thereof from N-acetylglucosamine or an activated form thereof, a non-natural amino acid analog of a natural amino acid to terminate formation of the chitin, an inhibitor of transcription of DNA which encodes an enzyme for producing the chitin or translation of RNA transcribed from the DNA, an inhibitor of an enzyme in a biosynthetic pathway producing the chitin from glucose, an inhibitor of a transaminase enzyme which produces an amino sugar from a keto sugar, a chitinase which degrades the chitin, or combination thereof. In a further embodiment, the composition comprises a compound selected from the group consisting of azaserine, acylurea, nikkomycin, polyoxin, polyene macrolide such as nystatin and mepartricine, and combinations thereof.

The present invention further provides a method for diagnosing a disease characterized by accumulation of congo red-staining plaques in a tissue of a mammal, preferably a human, which comprises detecting accumulated chitin or conjugate thereof in the tissue of the mammal.

In a further embodiment of the method, the disease is selected from the group consisting of spongiform encepalopathies, Alzheimer's disease, hemodialysis-related amyloidosis, primary systemic amyloidosis, secondary systemic amyloidosis, familial amyloid polyneuropathy I, familial amyloid polyneuropathy III, cerebral amyloid angiopathy, Finnish hereditary systemic amyloidosis, type II diabetes, injection-localized amyloidosis, medullary thyroid carcinoma, atrial amyloidosis, non-neuropathic systemic amylodosis, and hereditary renal amyloidosis.

In a preferred embodiment, the mammal is living and the chitin or conjugate thereof is detected based upon instrument controlled imaging of the chitin or conjugate thereof in the tissue of the mammal. In a further embodiment, the tissue is from a diseased mammal such tissue including tissue of the brain or the circulatory system.

In a further embodiment of the method, the disease is detected using a probe selected from the group consisting of a protein in a biosynthetic pathway for producing the chitin or conjugate thereof from glucose, DNA or RNA encoding a protein in a biosynthetic pathway for producing the chitin or conjugate thereof from glucose, and an antibody specific for the chitin or conjugate thereof or the disease is detected using a probe comprising a polypeptide fragment of a chitinase which binds the chitin or conjugate thereof without degrading the chitin or conjugate thereof.

The present invention further provides a method for reducing formation of chitin or conjugate thereof in a mammal which comprises administering an effective amount of a composition which inhibits formation of the chitin or conjugate thereof. In a further embodiment, the composition comprises an antibiotic, for example polyene macrolide antibiotics which includes nystatin and mepartricine. In a further embodiment, the composition comprises a compound selected from the group consisting of azaserine, acylurea, nikkomycin, polyoxin, and combinations thereof.

The present invention further provides a method for reducing chitin or conjugate thereof in a mammal which comprises administering an effective amount of a chitinase to the mammal. Preferably, the chitinase is human chitinase and the mammal is human.

### DESCRIPTION OF THE DRAWINGS

FIG. 1 Bio-Gel P10 Size exclusion chromatographs of neutral polysaccharides from AD and aged control brains. Brain tissues obtained at autopsy from 3 subjects with advanced AD (79-83 years; postmortem interval: 6-9 h) and 3 non-demented, age-matched control subjects (67-90 years; postmortem interval: 4.5-15 h) were delipidated with chloroform-methanol-water. Glycans were released by hydrazinolysis. Neutral glycans were separated by anion-exchange chromatography and labeled with aminobenzoic acid ethyl ester for UV detection at 314 nm. Polysaccharides were separated from oligosaccharides by Bio-Gel P4 chromatography. Polysaccharides were size-fractionated using Bio-Gel P10 chromatography (Column size: 120×1.0 cm. flow rate: 0.2 mL/min; 1.6 mL/tube). Chromatographs represent BIO-GEL P10 fractionated glycans from AD (solid dots) and control brain (open circles). Polysaccharides in the high molecular weight fractions are more highly enriched in AD than control tissues with average values of 4.1 mg/g and 1.5 mg/g of lyophilized brain tissue, respectively. The highest molecular weight peak from the AD brain (peak I) was further fractionated to give a component (>40,000 Daltons) containing exclusively amylose and another major component containing largely N-acetylglucosamine (7,000-40,000 Daltons). Peaks II and III also comprised largely N-acetylglucosamine.

FIGS. 2A shows 500 MHz ¹H-NMR spectra of polysaccharides from AD brains. Peaks II and III and the N-acetylglucosamine containing-fraction from re-chromatography of peak I were exchanged 3× with D₂O (99.996 atom % D). The ¹H NMR spectrum as obtained with a Varian VXR 500 spectrometer operating in the Fourier transform mode at a probe temperature of 50° C. Chemical shifts were expressed in ppm from acetone (2.225 ppm). FIG. 2A shows NMR spectra of Peak II with AD polysaccharides. The arrows indicate the resonances at 2.0 ppm, characteristic of an N-acetyl group. Spectra of the AD glycans display the same resonances as do the spectra from Peaks I and III from AD and control subjects (not shown).

FIGS. 2B shows 500 MHz ¹H-NMR spectra of polysaccharides from control brains as in FIG. 2A. FIG. 2B shows NMR spectra of Peak II with control polysaccharides. The arrows indicate the resonances at 2.0 ppm, characteristic of an N-acetyl group. Spectra of the control glycans display the same resonances as do the spectra from Peaks I and III from AD and control subjects (not shown).

FIG. 3A shows calcofluor-stained fibrils from AD brain tissue. Tissues from the same frozen brains used for biochemistry were immersion-fixed in 4% paraformaldehyde and cryostat-sectioned. Sections were incubated in 0.1% calcofluor/water for 1 h and then rinsed for 10 min before embedding in glycerol with anti-fade reagent (Molecular Probes). Calcofluor-stained chitin deposits were imaged on a Nikon Eclipse microscope using fluorescence optics (100 watt mercury illuminator, UV (Ex 340-380 nm, Em 4350485 nm)) with a 100× Plan Fluor objective (n.a. 1.3) with the CCD camera CoolSNAP_{fx} (Roper Scientific) on an ISEE Analytical Imaging station (ISEE Imaging Systems). FIG. 3A shows highly fluorescent diffuse plaques.

FIG. 3B shows calcofluor-stained fibrils from AD brain tissue prepared as in FIG. 3A. FIG. 3A shows cored plaques.

FIG. 3C shows calcofluor-stained fibrils from AD brain tissue prepared as in FIG. 3A. FIG. 3C shows two wispy fibrils not associated with plaques that were stained less brightly. The longer exposure time needed to image these single fibrils resulted in an increase of the background fluorescence. The black regions are freezing artifacts. These single fibrils differ from “neutrophil threads” due to tau aggregation by being more crystalline in appearance and less abundant than neutrophil threads.

FIG. 3D shows calcofluor-stained fibrils associated with a cortical blood vessel from tissue prepared as in FIG. 3A.

FIG. 3E shows calcofluor-stained fibrils associated with a cortical blood vessel from tissue prepared as in FIG. 3A. In this Figure, delipidated tissue from an AD brain was sequentially digested with DNAase, RNAase, amylase and protease. After dialysis and fractionation by sedimentation, the sample was further washed with water and concentrated. Blotting this sample on a slide and staining it with calcofluor revealed fibrils.

FIG. 3F shows granular calcofluor-stained deposits of undetermined origin in a section from a 55 year old control brain. Control sections and blots from enzymatically digested tissue not treated with calcofluor showed weak UV autofluorescence. Calcofluor staining was carried out on sections of frozen tissue not exposed to xylene because exposure of frozen sections to xylene enhanced this autofluorescence. Bar, 20 μm.

FIG. 4 shows analysis of water-insoluble brain glycans from AD tissue by Fourier transform infrared spectroscopy using microoptics. Tissue from an AD brain enzymatically digested as described in FIG. 3A-3F was analyzed with FT-IR spectroscopy. Signals typical of chitin at 3281cm⁻¹ (OH stretch), 2955, 2920 and 2849 (CH stretch), 1653 and 1647 (amide-1), and 1535 (amide-2) were observed. The spectrum matched that of chitin under the same conditions.

FIG. 5 shows analysis of water-insoluble brain glycans from AD tissue by gas chromatography-mass spectrometry. Tissue from an AD brain, enzymatically digested as described in FIGS. 3A was acetylized using 300 μL acetic acid, 300 μL acetic anhydride and 25 μL sulphuric acid at 100° C. for 20 h, neutralized with saturated NaHCO₃ and extracted with chloroform. GC-MS was performed with a DB-1 column at 160-230° C., 1° C./min. The mass spectrum is that of N-acetylglucosamine.

FIG. 6A shows bright field light micrograph of a commercial sample of chitin stained with congo red.

FIG. 6B shows polarized light micrograph of same field of FIG. 6A with an expanded inset on one set of fibrils and another inset from another field showing a yellow to yellow-green birefringence characteristic of amyloid plaque.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The present invention provides a method for detecting chitin and amyloid plaques which comprise chitin or a chitin core in mammals, including humans. The present invention further provides a method for diagnosing a disease caused by the accumulation of chitin or amyloid plaques comprising chitin or a chitin core in tissue of mammals, including humans, wherein the disease is characterized by the formation of congo red-staining bodies which are characteristic of amyloid plaques. The chitin or chitin core in the amyloid plaque makes detection and imaging of the amyloid plaque possible through means for detecting and imaging the chitin or chitin core in the amyloid plaque. The present invention further provides a method for preventing disorders or diseases in mammals, including humans, which are characterized by the formation and accumulation of chitin or amyloid plaques which comprise chitin or a chitin core. In particular, the present invention provides a method for treating a disorder or disease characterized by the accumulation of chitin or amyloid plaques which comprise chitin or a chitin core in a tissue of the mammal or human by administering to the mammal or human a composition comprising one or more molecules, chemicals, or drugs which inhibit formation of the chitin or chitin in the amyloid plaque, which effect a reduction in formation of the chitin or chitin in the amyloid plaque, or which effect a degradation of the chitin or chitin in the amyloid plaque.

The present invention is based upon the discovery disclosed herein of β-linked polymers of N-acetylglucosamine (chitin) and amylose in the brains of patients who had suffered from Alzheimer's disease (AD). The results presented in Example 1 are the first report of the presence of chitin in humans in which the chitin is associated with a disease characterized by formation of amyloid plaques. The discovery of high levels of soluble β-linked glucosamine-rich glycans in tissue from Alzheimer's-diseased brains had suggested to the inventors that insoluble glucosamine-containing polymers might also be present. Fibrils in senile plaques and blood vessels were isolated and shown to be stained in vitro with calcofluor, a carbohydrate-specific stain specific for β-1,4-linked polymers such as cellulose and chitin. The stain is not known to stain proteins. Actual fibrils were isolated from Alzheimer's disease brain by gravity sedimentation in water after exhaustive digestion of tissue with DNAase, RNAase, and amylases followed by exhaustive digestion with proteases. Fourier transform infrared spectroscopy on individual fibers using IR-microscopy produced signals which were consistent with signals produced by chitin and acetolysis of the fibers followed by GC-MS confirmed the presence of N-acetylglucosamine as the only component as would be expected for chitin. The isolated fibers and authentic chitin both stained identically with congo red displaying the same characteristic yellow to yellow green birefringence as do amyloid plaques. These results show that chitin forms a key component in the insoluble Alzheimer's plaque matrix which is also known to contain proteins. The chitin might provide a core or scaffold for the assembly of the amyloid proteins in forming the amyloid plaques. The chitin core or scaffold might be the basis for the extreme insolubility of the Alzheimer's plaque matrix. The presence of new glucosamine-rich glycans in the diseased state also has implications for developing immune responses for treating Alzheimer's disease and other diseases characterized by the presence of congo red-staining plaques. Therefore, the above findings provide the basis for developing novel strategies for detecting Alzheimer's disease and other diseases characterized by the presence of congo red-staining plaques and developing efficacious pharmaceutical and biological therapies for Alzheimer's disease and other diseases characterized by the presence of congo red-staining plaques.

The origin of the term “amyloid” to describe the lesions and plaques that characterize the brain in pathology of AD is not very clear but suggests a connection between the carbohydrate polymer amylose (starch) and the pathobiochemistry of the disease. Amyloid was introduced as a descriptive term in pathology by Virchow in 1854 (Virchow, Archiv. fuer. Pathol. Anat. und Physiol. und fuer klin. Med. 6: 135-137 (1854)), with the description of a substance found in the human brain and spinal cord that reacts chemically like cellulose based on pale blue staining with iodine and its violet appearance on the addition of sulfuric acid. He therefore named the stained material corpora amylacea (amyloid bodies) after starch. Only a few years later, Friedreich and Kekule determined in 1859 that no carbohydrate was present in these plaques and that they were comprised of protein although the descriptor ‘amyloid’ was retained (Friedreich and Kekule, Virch. Arch. Path. Anal. Physiol. 16: 50-65 (1859)). Amyloid plaque accumulation is not restricted to AD, but occurs in a variety of disease states and tissue types, and a number of proteins are associated with its accumulation. Fibrils that are red under transmitted light when stained with congo red but show yellow to yellow-green birefringence when the stained material is examined with plane polarized light is regarded as an identifying and common feature of all types of amyloid plaque. The discovery that chitin is associated with amyloid plaques in Alzheimer's-diseased brains suggests that chitin might serve as a scaffold for other amyloid plaques.

Amyloid or protein (fibril) plaque formation characterizes a large variety of diseases. These have been classified into 15 groups (Table 1). Amyloid (protein or fibril) plaque formation refers to an in vivo process in which one of the human amyloidogenic proteins abnormally self-assembles into a fibril 60-100 angstrom in width and of a variable length. The fibril has a characteristic cross-beta repeat structure where the individual beta strands composing the fibril are oriented perpendicular to the long axis of the fibril. The amyloidogenic proteins exhibit little sequence or structural homology, yet they are able to make amyloid fibrils of similar structure, as discerned from fiber X-ray diffraction patterns, their morphology in electron micrographs, and their ability to bind certain dyes (e.g. Congo Red) and exhibit birefringence. The staining of the plaques with congo red and the yellow to yellow-green birefringence of the congo red stain are common to all amyloid plaques. The present invention shows that polymers of N-acetyl glucosamine are an integral component of amyloid plaque and establish a biochemical basis for the link between glucose metabolism and amyloid plaque formation.

TABLE 1

| | Diseases with an amyloid plaque pathology | Amyloidogenic |
|---|---|---|
| | that can be stained with congo-red | protein source |
| CJD | spongiform encepalopathies | prion |
| APP | Alzheimer's | β-protein |
| HRA | hemodialysis-related amyloidosis | β-2-microglobin |
| PSA | primary systemic amyloidosis | Ig light chain |
| SAA 1 | secondary systemic amyloidosis | serum amyloid A |
| FAP I | familial amyloid polyneuropathy I | transthyretin |
| FAP III | familial amyloid polyneuropathy III | apolipoprotein A1 |
| CAA | cerebral amyloid angiopathy | cystatin C |
| FHSA | Finnish hereditary systemic amyloidosis | gelsolin |
| IAPP | type II diabetes | amylin |
| ILA | injection-localized amyloidosis | insulin |
| CAL | medullary thyroid carcinoma | calcitonin |
| ANF | atrial amyloidosis | atrial natriuretic |
| NNSA | non-neuropathic systemic amylodosis | lysozome |
| HRA | hereditary renal amyloidosis | fibrinogen |

Kelly, Curr. Opin. Struct. Biol. 6: 11-17 (1996)

The formation of amyloid plaques is the common link between diabetes, atherosclerosis, hypertension, Alzheimer's disease, and an entire host of other diseases as shown in table 1. The following summarizes the literature of several pathology studies which suggest a relationship between amyloid plaque formation and all of the diseases mentioned above.

Launer (Aging Res. Rev. 1: 61-77 (2002)) has presented evidence demonstrating a relationship between cardiovascular disease and Alzheimer's disease. Posner et al. (Neurol. 58: 1175-1181 (2002) have elucidated the relationship between hypertension in the elderly and Alzheimer's disease, vascular dementia, and cognitive function. O'Brien et al. (Lancet Neurol. 2: 89-98 (2003)) have reviewed the link between cardiovascular impairment and Alzheimer's disease. A case for treating Alzheimer's as primarily a vascular disease has been made by Zlokovic (Adv. Drug Delivery Rev. 54: 1553-1559 (2002)).

Several epidemiological studies suggest that there might be a relationship between diabetes and dementia. These studies include Peila et al. (Diabetes 51: 1256-1262 (2002)), Bruce et al. (Diabetes Res. Clin. Prac. 53: 165-172 (2001)), Grodstein et al. (Diabetes Care 24: 1060-1065 (2001)).

Several biochemical studies such as the one by Awad et al. (Behavioral Neurosci. 116: 691-702 (2002)) and Gasparini et al. (J. Neurosci. 21: 2561-2570 (2001)) suggest a relationship between glucose regulation or insulin regulation of glucose and Alzheimer's disease.

Several biochemical studies such as those by Bigl et al. (J. Neural Trans. 110: 77-94 (2003); J. Neural Trans. 106: 499-511 (1999)), which show that glycolysis is impaired in cells peripheral to amyloid plaques, suggest a relationship between glycolysis and Alzheimer's disease. Positron emission tomography using ¹⁹F 2-fluoro-2-deoxyglucose has shown that tissue in Alzheimer's diseased brain is impaired in the metabolism of glucose (Pietrini et al., Intl. J. Psychophysiol. 37: 87-98 (2000)). Glucose transport in Alzheimer's diseased brain is also impaired because of a reduction in the number of glucose transporter proteins (GLUT1 and GLUT3) (Harr et al., J. Neuropathol. Exp. Neurol. 54: 38-41 (1995); Mooradian et al., Neurobiol. Aging 18: 469-474 (1997)). The insulin signaling cascade in the brains of patients with Alzheimer's disease is also known to be impaired (Frolich et al., Annals of the New York Acad. Sci. 893: 290-293 (1999)).

Several clinical, pathological and biochemical studies and analyses such as in Serpell et al. (Cell. Molec. Life Sci. 53: 871-887 (1997)) reinforce the idea that there is a relationship between amyloid plaque formation in Alzheimer's disease and amyloid plaque formation in prion-related encephalopathies such as bovine spongiform encephalopathy (BSE) and Creutzfeldt-Jakob disease (CJD). The amyloid diseases have been summarized in Kelley (Curr. Opin. Struct. Biol. 6 11-17 (1996)).

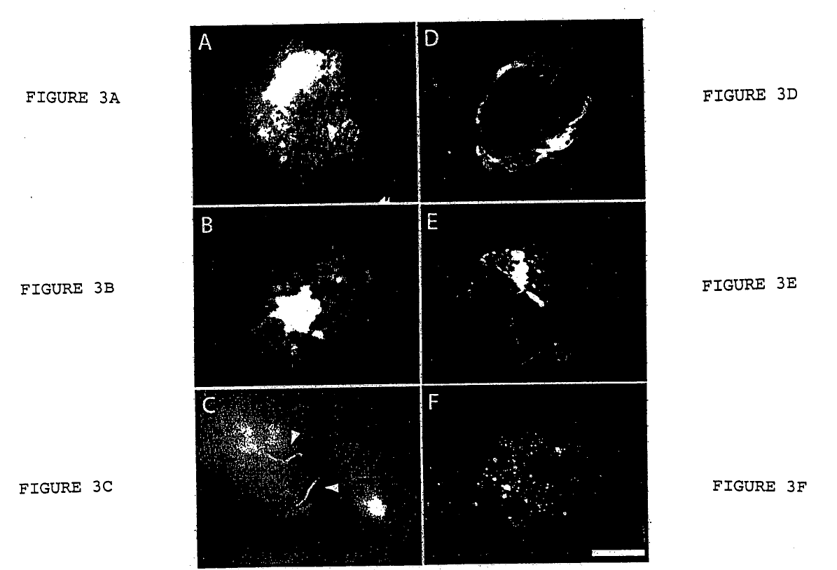

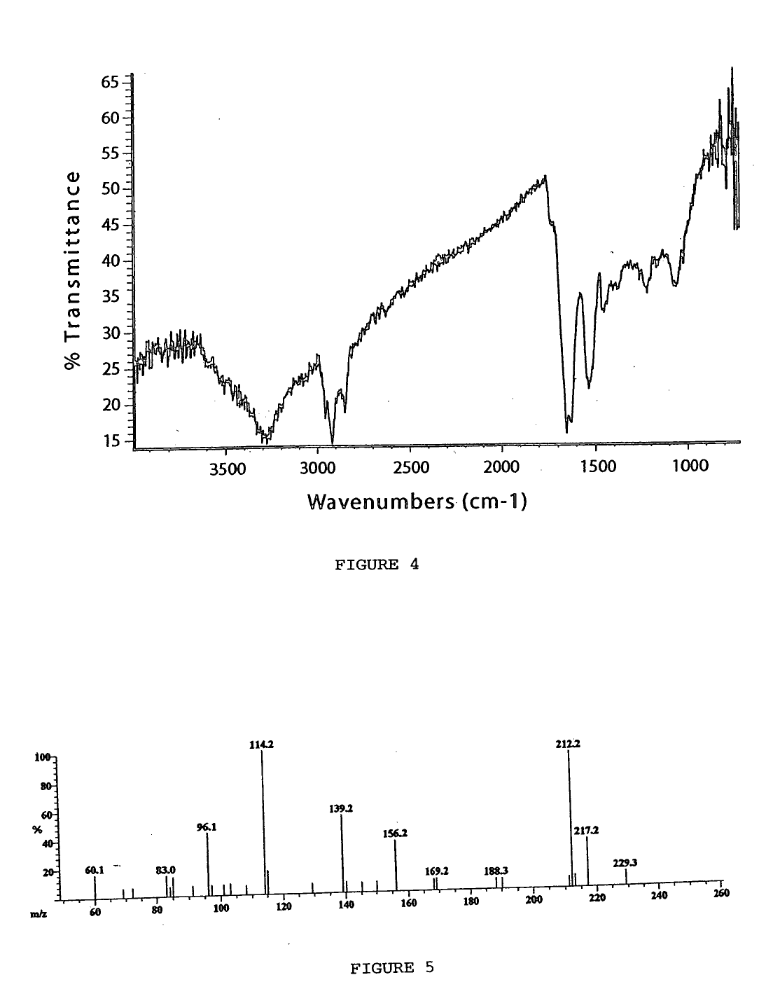

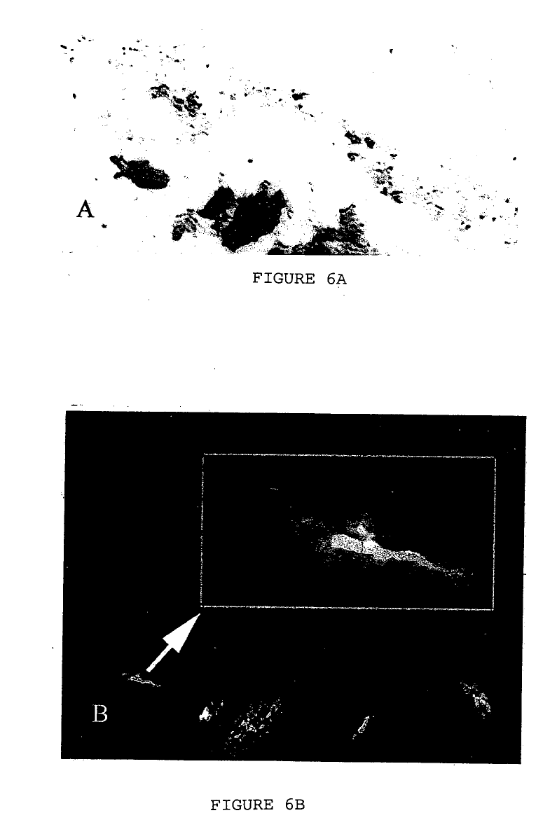

It is demonstrated herein using microscopy with specific labels that a chitinaceous substance is present in amyloid plaques and in blood vessels of persons who suffered from Alzheimer's disease. As shown by isolation and rigorous chemical proof, the material is chitin. Proof includes: (1) rigorous isolation and purification of fibrils from Alzheimer's disease amyloid plaques and staining with calcofluor which showed that the fibrils have the same microscopic and cytochemical properties as those observed in intact amyloid plaques when stained in situ (FIGS. 3A-3F); (2) Fourier transform IR spectroscopy on isolated, purified fibrils which produced signals typical of chitin and which matched a sample of authentic chitin under similar conditions (FIG. 4); (3) chemical degradation of purified fibrils, by acetolysis followed by chemical analysis of the degradation products by gas chromatography and mass spectrometry showed that the fibrils comprised per-acetylated glucosamine (FIG. 5); and, (4) light microscopy showing that chitin has the same cytochemical attributes as amyloid plaques such as staining red with congo red and having a yellow to yellow-green birefringence (FIG. 6B).

Amyloid plaque formation characterizes all of the diseases shown in Table 1. Because chitin is insoluble, a plaque comprising chitin forms an impedance or barrier to the flow of blood and the delivery of oxygen and nutrients to tissue peripheral or adjacent to the chitin-containing plaque. The chitin and the chitin-comprising plaques would also serve as a matrix on which further serum protein deposition can occur. Chitin build-up and protein build-up would trap lipids and other aggregates or macromolecules in circulation in blood. This reduction or restriction in blood flow would result in an increase in blood pressure and a loss of oxygen to peripheral tissue. As a result, clinical manifestations would include dementia because of high blood pressure and cell death in the peripheral or adjacent tissue. The deposition of chitin in blood vessels would also lead to hardening of the arteries and to a loss of flexibility in the arteries resulting in high blood pressure, stroke, and other cardio-vascular problems. The deposition of chitin on the walls of blood vessels can be seen in FIG. 3D. The Figure shows cross-sections of blood vessels from the brains of Alzheimer's disease patients stained with calcofluor. The blood vessel cross-sections have bright circular rings of stain on the inner walls of the blood vessels indicating the formation of a chitin scale or layer on the inner walls of the blood vessels.

In the prior art, the current understanding is that the fibrils in amyloid plaques consist exclusively of proteins and that carbohydrates are not a component of the fibrils in the amyloid plaques. In addition, the prior art is of the view that chitin is not found in humans and other higher organisms. Therefore, the synthesis or degradation of chitin has not been pursued as a therapeutic strategy for treating diseases other than particular fungal infections. The current drug targets for Alzheimer's disease have been recently reviewed by Lahiri et al. (Curr. Drug Targets 4: 97-112 (2003)) and none involve regulation of the biosynthesis and metabolism of glucose, glucosamine, or chitin. Here, it is shown that the formation of amyloid plaques revolve around the presence of chitin or chitin scaffolds or cores and the control and treatment of diseases broadly referred to as amyloidosis include means for inhibiting formation of or reducing the presence of chitin or chitin scaffolds or cores. The treatment can further include means for inhibiting various components and pathways in glucose metabolism, in particular, the biosynthesis of glucosamine from glucose and its conversion to chitin, and more particularly, inhibiting particular key steps and controls in the biosynthesis of glucosamine from glucose and it conversion to chitin. The biosynthetic pathway for chitin biosynthesis is shown in Scheme 1. Points A, B, and C in the pathway would be effective points for exerting control of chitin biosynthesis. This is a new direction that enables therapeutic approaches that are not suggested by the current prior art.

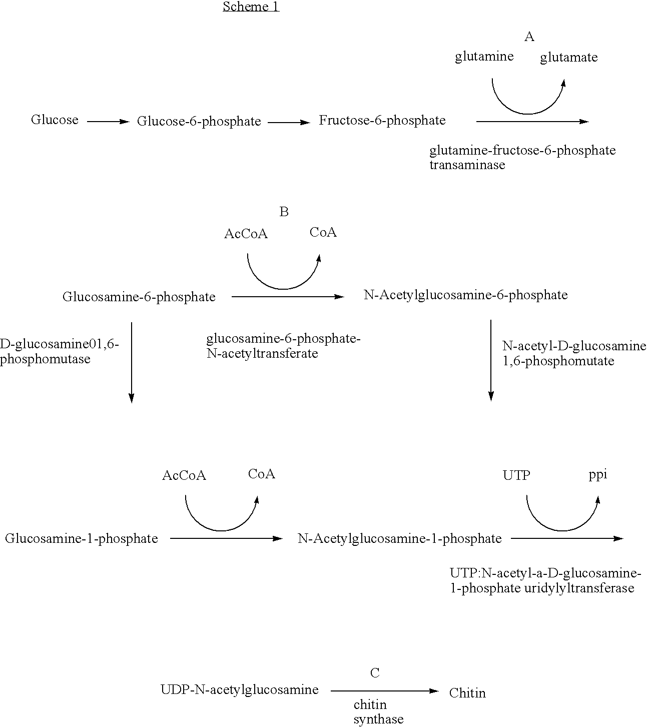

The biosynthetic pathway for glucosamine and eventually chitin from glucose shown in Scheme 1 is well established. Chitin synthase is the enzyme that converts UDP-N-acetylglucosamine to chitin. It is known that chitin synthase activity is induced allosterically by high levels of glucosamine (Horst M et al., Eur. J. Biochem. 237: 476-482 (1996); Horst and Rast, In RAA Muzzarelli (ed): Chitin Enzymology. European Chitin Society, Ancona, pp. 47-56 (1993)) making the regulation of glucosamine synthesis a key prospect for controlling chitin levels as a therapeutic strategy in amyloid diseases. Sequences of over 70 chitin synthases have been compared across insects and fungi and the critical catalytic sites are highly conserved making it possible to predict the fold of the catalytic domain across sequences (Horsch and Sowdhamini, In Muzzarelli (ed): Chitin Enzymology vol 2. Atec Edizioni, Italy pp. 447-448 (1996)). Genes encoding hyaluronic acid synthase have been identified in humans and appear to be similar in structure to chitin synthases (Recklies, Biochem. J. 354: 17-24 (2001)). Normally, these chitin synthase-like synthases produce hyaluronic acid from UDP-N-acetylglucosamine and UDP-glucuronic acid. However, in the absence of UDP-glucuronic acid, these chitin synthase-like synthases can produce chitin from UDP-N-acetylglucosamine (Yoshida et al., J. Biol. Chem. 275: 497-506 (2000)). Because of the highly conserved homologies between the various chitin synthases, any of the structure-based or mechanism-based chitin synthase inhibitors that have been developed for yeast, fungal, bacterial or insect control can potentially be used in man.

Chitinases have been discovered in mammals, including humans, and have been described in U.S. Pat. No. 6,399,571 to Gray et al. and U.S. Pat. Nos. 6,057,142 and 6,301,118 to Aerts. Human chitinase with chitotriosidase activity is expressed by phagocytes (macrophages). A similar chitinase has been found in the lung and an acidic chitinase has been found in the intestine. Chitinases are thought to provide a defense against opportunistic infections by fungi and bacteria. Chitinases may also be involved in removing chitin which may be formed by fluctuations in the ratios of UDP-N-acetylglucosamine and UDP-glucuronic acid in the synthesis of hyaluronic acid. Chitin which is not degraded might accumulate in the body and provide a scaffold or core for assembly of amyloidogenic proteins such as β-protein of Alzheimer's disease into amyloid plaques. Therefore, mutations which cause a decrease or cessation of chitinase activity may be involved in the formation of amyloid plaques because the mutations allow for an accumulation of chitin either from fungal or bacterial infections, from defects in the synthesis of hyaluronic acid which shifts synthesis from hyaluronic acid towards chitin, defects in an exogenouse (bacterial or fungal) or endogenous (not yet discovered in mammals or humans) pathway for the synthesis of chitin which result in an excess accumulation of chitin, or fluctuations in the ratio of UDP-N-acetylglucosamine and UDP-glucuronic acid in the pathway for synthesizing hyaluronic acid which shifts synthesis towards chitin. The chitin then serves as a scaffold for the assembly of amyloid plaques, the assembly of which occurs because of yet unknown defects which cause the particular amyloidogenic proteins comprising the amyloid plaques to self-assemble on the chitin scaffold into the amyloid plaques.

Therefore, the detection methods set forth below include detecting chitin and chitin conjugates which accumulate in the mammal or human as a result of a mutation in the hyaluronic synthase or chitinase, a fluctuation in the ratio of UDP-N-acetylglucosamine and UDP-glucuronic acid in the pathway for synthesizing hyaluronic acid which shifts synthesis towards chitin, a mutation in another enzyme involved in the pathway for synthesis of hyaluronic acid which results in the synthesis of chitin, a mutation in an enzyme in a pathway for synthesizing chitin in the mammal or human which results in overproduction of chitin, or chitin produced by fungi or bacteria. The treatment methods set forth below include inhibiting synthesis of or degrading chitin and chitin conjugates which accumulate in the mammal or human as a result of a mutation in the hyaluronic synthase or chitinase, a fluctuation in the ratio of UDP-N-acetylglucosamine and UDP-glucuronic acid in the pathway for synthesizing hyaluronic acid which shifts synthesis towards chitin, a mutation in another enzyme involved in the pathway for synthesis of hyaluronic acid which results in the synthesis of chitin, a mutation in an enzyme in a pathway for synthesizing chitin in the mammal or human which results in overproduction of chitin, or chitin produced by fungi or bacteria. The treatments can further include methods which degrade the amyloidogenic proteins in the amyloid plaques as well.

Detection of Diseases Characterized by Accumulation of Chitin

Because chitin or chitin conjugate thereof appears to provide a scaffold or core for assembly of amyloidogenic proteins into congo red-staining amyloid plaques, the presence of chitin in a human is an early indicator that the amyloid plaques will or are in the process of being formed in the human. Thus, a diagnostic assay for detecting the presence of chitin in humans provides an early detection means for identifying those persons who are predisposed to or in the process of forming amyloid plaques. The detection of chitin before the development of amyloid plaques or a significant number of amyloid plaques enables treatment strategies to be developed for preventing the adverse effects associated with the diseases which are associated with amyloid plaques such as CJD (spongiform encepalopathies), APP (Alzheimer's disease), HRA (hemodialysis-related amyloidosis), PSA (primary systemic amyloidosis), SAA 1 (secondary systemic amyloidosis), FAP I (familial amyloid polyneuropathy I), FAP III (familial amyloid polyneuropathy III), CAA (cerebral amyloid angiopathy), FHSA (Finnish hereditary systemic amyloidosis), IAPP (type II diabetes), ILA (injection-localized amyloidosis), CAL (medullary thyroid carcinoma), ANF (atrial amyloidosis), NNSA (non-neuropathic systemic amylodosis), and HRA (hereditary renal amyloidosis).

Methods for detecting chitin polymers and amyloid plaques in vivo include computed tomography, magnetic resonance imaging or nuclear magnetic resonance (NMR), ultrasound, and related methods. ¹⁸F or ¹⁹F, ²H, ³¹P, ²³Na, ¹⁴N, and ¹³C isotopes for labeling compounds in the biosynthetic pathway, such as N-acetylglucosamine. For instance, when the fluoro-labeled N-acetylglucosamine is utilized to produce chitin concentrations in the brain, it can be detected by magnetic resonance imaging or computed tomography in the brain or other tissues. Other compounds in the biosynthetic pathway of chitin can also be labeled.

For example, in a first computed tomography or related method, the patient is administered ¹⁸F-fluorinated forms of glucosamine or N-acetylglucosamine. The ¹⁸F-labeled material is incorporated into the chitin polymers which renders both chitin polymers without amyloid proteins assembled thereon (pre-plaques) and amyloid plaques (in various stages of development) in which the amyloid proteins have been assembled on the chitin polymers visible by positron emission computed tomography or the like. Even though current instrumentation is limited by spatial resolution and sensitivity, advancements over the life of current patients will make this method possible. For example, one method with a resolution of less than 10 μm is optical coherence tomography.

In a first magnetic resonance imaging method, the patient is administered over extended periods spin-labeled (or other paramagnetic form) glucose, glucosamine, fructose, N-acetylglucosamine, or some other natural or unnatural precursor of chitin. The spin-label is incorporated into the chitin polymer which renders both chitin polymers without amyloid proteins assembled thereon (pre-plaques) and amyloid plaques (in various stages of development) in which the amyloid proteins have been assembled on the chitin polymers visible by magnetic resonance imaging.

In a second magnetic resonance imaging method, the patient is administered over extended periods ¹⁹F-fluorinated forms of glucose, glucosamine, fructose, N-acetylglucosamine, or some other natural or unnatural precursor of chitin. The ¹⁹F-labeled material is incorporated into the chitin polymers which renders both chitin polymers without amyloid proteins assembled thereon (pre-plaques) and amyloid plaques (in various stages of development) in which the amyloid proteins have been assembled on the chitin polymers visible by magnetic resonance imaging.

The present invention can also use labeled chitin binding fragments or degrading proteins as probes to detect chitin directly. Probes specific for chitin include chitin binding lectins such as chitovibrin which is disclosed in U.S. Pat. Nos. 5,914,239 and 6,121,420, both to Laine; chitin binding fragments derived from human chitinase as disclosed in U.S. Pat. Nos. 6,399,571, 6,200,951 and 6,372,212, all to Gray et al.; chitin binding fragments derived from chitinases isolated from plants such as Arabidopsis thaliana (Samac et al., Plant Physiol. 93: 907-914 (1990), tobacco (Lawton et al., Plant Mol. Biol. 19: 735-743 (1992)), fungi such as yeast (McCreath et al. Yeast 12: 501-504 (1996)), bacteria such as Bacillus circulans (Watanabe et al., J. Bacteriol. 1 74:408-414 (1992)), mammals, and insects. Chitin synthase which appears to be closely related to or substantially identical to human hyaluronic acid synthase (U.S. Pat. No. 6,492,150 to McDonald et al.), can be labeled and detected by tomography or NMR in vivo or in vitro. Polyclonal antibodies, monoclonal antibodies, Fab fragments, recombinant Fab polypeptides, Fv fragments, recombinant single-chain Fv polypeptides, and variations thereof which are specific for chitin can also be used as a probe. Anti-chitin antibodies have been disclosed in U.S. Pat. No. 5,004,699 to Winters. These probes can be labeled as above for tomographic or magnetic resonance imaging or with a relaxation agent such as a paramagnetic transition metal species which would enable magnetic resonance imaging by contrasting. The labeled probes can be provided intravenously or injected directing into the area of the patient to be diagnosed.

Ultrasound methods include methods such as that disclosed in U.S. Pat. No. 6,521,211 B1 to Unger et al. which can be adapted to use the above chitinase binding probes and antibodies as the targeting ligand.

An alternative method for detecting chitin uses anti-chitin polypeptide antibodies or derivatives thereof, preferably scFv polypeptides, displayed on filamentous bacteriophage as a probe for chitin and amyloid plaques containing chitin. The probes are administered intravenously, intranasally, intramuscularly, or the like. Preferably, the probes are labeled with a radioisotope or contrast agent as above for computed tomography, magnetic resonance imaging, ultrasound, and the like. A similar method for detecting amyloid plaques in mammals using anti-amyloid scFv polypeptides is disclosed in published U.S. Patent Application No. 20020052311 A1 to Solomon and Frenkel and in Frenkel and Solomon, Proc. Natl. Acad. Sci. USA 99: 5675-5679 (2002). The methods may be adapted for detecting chitin by substituting the anti-amyloid scFv therein with the anti-chitin scFv taught herein.

The present invention can also use labeled chitin binding fragments or degrading proteins to detect chitin directly as described in U.S. Pat. No. 6,399,577 to Gray et al. as well as the related U.S. Pat. Nos. 6,200,951 and 6,372,212. Chitin synthase (closely related to or substantially identical to human hyaluronic acid synthase and described in U.S. Pat. No. 6,492,150 to McDonald et al.), can be detected NMR in vivo or in vitro.

Various labeled molecules, chemicals, or drugs which interfere with chitin formation by inhibiting chitin synthase can be used, such as antibiotics which interfere with chitin synthase as described in U.S. Pat. No. 5,330,976 to Hecter et al.

Treatment for Diseases Characterized by Accumulation of Chitin

Once chitin or conjugate thereof and/or congo red-staining amyloid plaques have been identified to be present in the mammal or human, then treatment of the disease is achieved by control of the rate of accumulation, destruction of the accumulated chitin, or inhibition of chitin accumulation. Control, destruction, or prevention of chitin accumulation can be achieved by one or more biochemical means which affect one or more enzymes involved in the synthesis or degradation of the chitin or one or more immunological means which affect the formation of chitin, the structure of the chitin, the deposition of amyloid protein on the chitin scaffold, or one or more of the enzymes involved in the formation of the chitin, or a combination of one or more biological and one or more immunological means. Because chitin appears not to endogenous to normal mammals and humans, the immunological means can be a vaccine comprising antibodies against chitin. A vaccine is useful for the prophylactic treatments for mammals, particularly mammals predisposed to developing a disease characterized by amyloid plaques and the accumulation of chitin.

The exertion of control at specific points of the biochemical pathway from glucose to glucosamine to control chitin synthesis or deposition as a general therapeutic strategy for treating or staving off (in a prophylactic approach) amyloid diseases is a general objective of this invention. Such diseases include CJD (spongiform encepalopathies), APP (Alzheimer's disease), HRA (hemodialysis-related amyloidosis), PSA (primary systemic amyloidosis), SAA 1 (secondary systemic amyloidosis), FAP I (familial amyloid polyneuropathy I), FAP III (familial amyloid polyneuropathy III), CAA (cerebral amyloid angiopathy), FHSA (Finnish hereditary systemic amyloidosis), IAPP (type II diabetes), ILA (injection-localized amyloidosis), CAL (medullary thyroid carcinoma), ANF (atrial amyloidosis), NNSA (non-neuropathic systemic amylodosis), and HRA (hereditary renal amyloidosis).

For example, in one embodiment, inhibiting synthesis of chitin and thus, inhibiting formation of amyloid plaques, is achieved by reversing one or more steps in the biosynthesis pathway leading to chitin. Without the chitin, the amyloid plaques cannot form. Inhibition can be achieved by altering the flux of specific metabolites through the biosynthetic pathway. This therapeutic strategy is enabled by the results and arguments disclosed herein.

In a second embodiment, the chitin synthase-like enzyme is specifically inhibited. The inhibition can be by the antibiotics or other chemicals which attack the chitin synthase, such as those described by Hecter et al. in U.S. Pat. No. 5,330,976, particularly the Nikkomycins and Polyoxins and micronazoles. In general, any one or more of the antimycotic agents which attack the biosynthetic pathway of chitin can be used. Alternatively, the chitin binding fragments of U.S. Pat. No. 6,200,951 to Gray et al. can be linked with chitinase or other chemicals which degrade or interfere with the synthesis of chitin. In this instance the formation of chitin or the degradation of chitin can be achieved.

In a third embodiment, the chitin is degraded with degradation enzymes such as chitinases. For example, U.S. Pat. Nos. 6,200,951, 6,399,571, and 6,372,212 to Gray et al., describes a human chitinase, the DNA encoding the chitinase, and fragments of the chitinase for detecting chitin, binding chitin, and treating fungal infections. These patents provide a detailed background regarding chitinase which enables the present invention.

Other specific strategies for preventing chitin accumulation and thus, formation of amyloid plaques include (1) blocking the activity of chitin synthases by transition state inhibitors such as the nucleoside peptides Nikkomycins and polyoxins disclosed in U.S. Pat. No. 5,330,976 to Hector et al. and other fungal inhibitors which block the activity of chitin synthases; (2) blocking the activity of chitin synthases by transition state inhibitors to N-acetylglucosamine donor-acceptor complexes; (3) blocking the synthesis of messenger RNA for chitin synthases from the DNA template using transcription inhibitors which preferentially bind to the regulatory sequences for the gene encoding the chitin synthase thereby down-regulating expression of the gene; (4) blocking the synthesis of chitin synthases from the RNA template using translation inhibitors or antisense technology or the like to preferentially bind the RNA template and preferably degrade the template; (5) using transcription enhances which preferentially enhance transcription of genes encoding chitinases, (6) regulating the levels of glucosamine by controlling the synthesis and build up of fructose-6-phosphate; (7) regulating the levels of fructose-6-phosphate by controlling the levels of glucose; (8) regulating the levels of glucosamine by regulating the amination of fructose-6-phosphate to glucosamine-6-phosphate by regulating the activity of the enzyme by inhibitors such as azaserine which inhibits the enzyme glutamine:fructose-6-phosphate aminotransferase; (9) regulating the levels of glutamine thus regulating amination of fructose-6-phosphate; (10) incorporation of chain terminators into chitin chains; (11) general chitin synthesis inhibition by reagents such as acylureas; and (12) inhibiting chitin synthases by binding the chitin synthases with polyene macrolide antibiotics such as nystatin and mepartricine B.

The above chitin compositions comprising chemicals, molecules, or drugs which inhibit synthesis or accumulation of chitin, regulate synthesis or accumulation of chitin, or degrade or reduce accumulation of chitin can be administered to mammals and human patients by methods which include, but are not limited to, intramuscular, intraperitoneal, intradermal, subcutaneous, intravenous, intra-arterial, intraocular, and oral as well as transdermal or by inhalation or suppository. The preferred routes of administration include intranasal, intramuscular, intraperitoneal, intradermal, and subcutaneous injection. The composition can be administered by means including, but not limited to, syringes, needle-less injection devices, or microprojectile bombardment gene guns (biolistic bombardment).

The compositions are preferably formulated in pharmaceutically acceptable carriers according to the mode of administration to be used. One skilled in the art can readily formulate a composition that comprises one or more of the above compositions. In cases where intramuscular injection is preferred, an isotonic formulation is preferred. Generally, additives for isotonicity can include sodium chloride, dextrose, mannitol, sorbitol, and lactose. In particular cases, isotonic solutions such as phosphate buffered saline are preferred. The formulations can further provide stabilizers such as gelatin and albumin. In some embodiments, a vasco-constriction agent is added to the formulation. The pharmaceutical preparations according to the present invention are provided sterile and pyrogen free. However, it is well known by those skilled in the art that the preferred formulations for the pharmaceutically acceptable carrier which comprise the compositions are those pharmaceutical carriers approved in the regulations promulgated by the United States Food and Drug Administration, United States Department of Agriculture, or equivalent government agency in a foreign country such as Canada or Mexico, for compositions. Therefore, the pharmaceutically acceptable carriers for commercial production of the compositions are those carriers that are already approved or will at some future date be approved by the appropriate government agency in the United States of America or foreign country.

An example for treating human patients with atrial amyloidosis or cerebral amyloid angiopathy includes administering one or more of the above compositions to the patient intravenously. For example, the patient can be administered a chitinase such as has been disclosed in U.S. Pat. Nos. 6,399,571, 6,200,951 and 6,372,212, all to Gray et al., or U.S. Pat. Nos. 6,057,142 and 6,303,118, both to Aerts, an inhibitor of transamination of keto sugars to amino sugars, or antibodies specific for the chitin or chitin conjugate. In particular embodiments, the composition can further include one or more inhibitors of self assembly of amyloidogenic proteins such as antibodies against those sites on the amyloidogenic protein which enable aggregation of the proteins into a plaque. An example of such an inhibitor includes antibodies against the EFRH epitope of β-amyloid as disclosed in Frenkel et al., Proc. Natl. Acad. Sci. USA 97: 11455-11459 (2000) or a compound which specifically degrades the amyloidogenic protein comprising the plaques.

Administering compositions to a patient or animal for treating brain diseases requires getting the composition past the blood-brain barrier. This can be achieved using invasive means such as surgical intervention or non-invasive means. Therefore, for administering many of the above composition to the brain for the treatment of Alzheimer's disease and other neurological diseases which are characterized by amyloid plaques such as the spongiform encephalopathies, it is preferable to be able to target the composition to the brain in a non-invasive manner. A non-invasive means is desirable and advantageous because it is expected that in many cases, repeated administrations of the composition is likely. Therefore, it is desirable that the composition be administered by a route that is no more invasive than a simple intravenous injection. With this approach, the composition is delivered through the blood-brain barrier (BBB) by targeting the composition to the brain via endogenous BBB transport systems. Carrier-mediated transport systems exist for the transport of nutrients across the BBB. Similarly, receptor-mediated transcytosis systems operate to transport circulating peptides across the BBB, such as insulin, transferrin, or insulin-like growth factors. These endogenous peptides can act as transporting peptides to ferry drugs and the like across the BBB. In this approach, the drug that is normally not transported across the BBB is conjugated to a transportable peptide and the drug/transportable peptide conjugate undergoes receptor-mediated transcytosis through the BBB (See for example U.S. Pat. No. 4,801,575 to Pardridge).

Thus, for example, a chitinase is conjugated to a transport peptide such as insulin. The insulin enables the chitinase to cross the BBB where it able to then migrate to those areas of the brain which have chitin accumulated thereat such as amyloid plaques and degrade the chitin. Degrading the chitin results in the dissolution of the amyloid plaques and the prevention of assembly of the amyloidogenic proteins on the chitin to form amyloid plaques. In some embodiments, it is desirable to include with the chitinase coupled to a transport peptide an amyloidogenic protein degrading compound or antibody which inhibits amyloid aggregation coupled to a transport peptide. The combination enables both the amyloid aggregates to be degraded and the chitin to be degraded. Suitable chitinases are disclosed in as disclosed in U.S. Pat. Nos. 6,399,571, 6,200,951 and 6,372,212, all to Gray et al., and U.S. Pat. Nos. 6,057,142 and 6,303,118, both to Aerts.

U.S. Pat. No. 6,372,250 to Pardridge discloses an improved method for transporting the above therapeutic compounds across the BBB which uses liposomes which contain the therapeutic compound and which has disposed in the lipid membrane a plurality of agents which enable the liposomes to cross the BBB. These agents include insulin, transferrin, insulin-like growth factor, leptin, and low density lipoproteins. Alternatively, the agent is a peptidomimetic antibody which mimics the preceding peptides and which binds the receptor for the above proteins. The lipid membrane preferably further includes targeting agents which targets the liposome to the cells in the brain involved in synthesizing the chitin or the amyloid plaques or to the chitin or amyloid plaques per se. For example, the targeting agent can include the chitin binding sites of the chitinase identified in chitin binding lectins such as chitovibrin which is disclosed in U.S. Pat. Nos. 5,914,239 and 6,121,420, both to Laine; chitin binding fragments derived from human chitinase as disclosed in U.S. Pat. Nos. 6,399,571, 6,200,951 and 6,372,212, all to Gray et al., or U.S. Pat. Nos. 6,057,142 and 6,303,118, both to Aerts; chitin binding fragments derived from chitinases isolated from plants, fungi, bacteria, mammals, and insects. Alternatively, the targeting agent can include polyclonal antibodies, monoclonal antibodies, Fab fragments, recombinant Fab polypeptides, Fv fragments, recombinant single-chain Fv polypeptides, and variations thereof which are specific for chitin. In particular embodiments, the therapeutic compound can further include inhibitors or degraders of amyloid plaques.

Thus, for example, the liposome contains a chitinase and the lipid membrane includes a transport peptide such as insulin and an agent which targets chitin or amyloid proteins or both. The insulin enables the liposome to cross the BBB and the agent targets the liposome to chitin or amyloid plaques wherein the chitinase is available to degrade the chitin. In some embodiments, it is desirable to include with the chitinase an amyloidogenic protein degrading compound or antibody which inhibits amyloid aggregation coupled to a transport peptide. The combination enables both the amyloid aggregates to be degraded and the chitin to be degraded. In other embodiments, the liposome contains amyloidogenic protein degrading compounds without the chitinase.

Other methods for getting chitinases, antibodies, and/or inhibitors across the BBB include the filamentous phage intranasal delivery method disclosed in Frenkel and Solomon, Proc. Natl. Acad. Sci. USA 99: 5675-5679 (2002).

Pharmaceuticals and Nutraceuticals for Treating or Inhibiting Diseases Characterized by Amyloid Plaque Formation.

Various nutraceuticals can be used to redirect the synthesis of chitin. These include sugar uronic acids which direct the synthesis towards hyaluronic acid or other natural polycarbohydrate polymers, pectin, condroitin sulfate and non-natural sugar uronic acids (isomer) can be used in this manner. For example, see Yoshida et al. (J. Biol. Chem. 275: 497-506 (2000) which showed that the mouse analogue to the human enzyme for synthesizing hyaluronic acid from N-acetylglucosamine and glucuronic acid will make chitin when provided only N-acetylglucosamine. Therefore, providing glucuronic acid or precursors to glucuronic acid using any of the previously described methods will shift the synthesis from chitin back to hyaluronic acid in those cases where glucuronic acid is absent or where N-acetylglucosamine is in excess.

However, before treating a patient or animal with any of the above products, it is desirable to detect and monitor the chitin so that the effectiveness of the treatment can be monitored. In this connection, the use of glucosamine or N-acetylglucosamine as a nutraceutical is clearly contraindicated for those with a possibility of Alzheimer's disease or any of the other diseases characterized by the formation amyloid plaques, particularly those diseases which are inheritable.

Prophylactic Methods for Inhibiting Diseases Characterized by Formation of Amyloid Plaques

The above chitin synthesis inhibitors can also be used as a prophylactic means in humans to protect humans from diseases that are characterized by formation of amyloid plaques, particularly humans predisposed to amyloid plaque formation, and in the cattle industries to protect cattle against bovine spongiform encephalopathy (Mad cow disease). These inhibitors can be administered on a continual basis or on an intermittent basis.

Since chitin does not naturally occur in mammals and humans in the absence of a fungal or bacterial infection, animals and humans can be vaccinated with a chitin or chitin conjugate to stimulate an immune response to the chitin or conjugate. The immune response would include production of antibodies which would bind to chitin or conjugate wherever it might occur in the animal or human. The immune response might also include a cell-mediated response which would include macrophages which are capable of digesting the chitin. Human macrophages are known to contain a chitotriosidase (chitinase) activity. The immune response would protect the animal or human against diseases characterized by congo red-staining amyloid plaques because as the chitin is being formed, the vaccinated animal or human would be producing antibodies and macrophages in response. The antibodies and macrophages would lead to the degradation and removal of the chitin before it can lead to the formation of amyloid plaques.

The above vaccines for inducing an immune response against chitin can be administered in conjunction with chitin synthase inhibitors and pharmaceuticals and nutraceuticals which inhibit the synthesis of chitin.

The following examples are intended to promote a further understanding of the present invention.

### EXAMPLE 1

This example reports the discovery of chitin in the brains of patients who had died of Alzheimer's disease and the connection of the chitin with the fibrils comprising the amyloid plaques in the brain.

During the course of investigations on the alteration of glycosylation of neurons with development and differentiation of the nervous system the inventors embarked on a study of changes that accompany degenerative pathways. It was reasoned that if glycosylation were a defining feature of neuronal development and health there should be changes that define the pathology of neurodegenerative diseases.

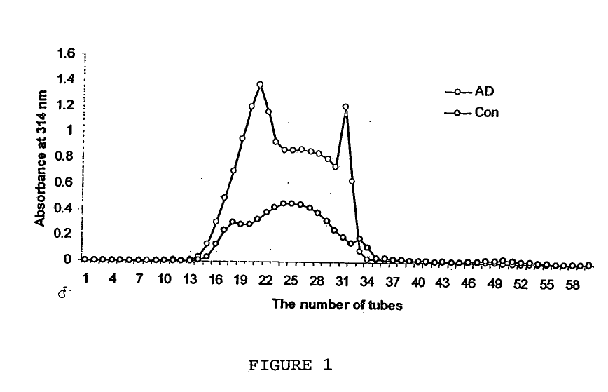

To this end, the inventors examined the glycosylation of brain tissue obtained at autopsy from subjects with AD (Braak stage V-VI) (Braak and Braak, Acta Neuropathol. (Ber) 82: 239-259 (1991)) and age-matched subjects with no evidence of dementia. The tissue was obtained from the University of Maryland Brain and Tissue Bank. The material was delipidated with chloroform-methanol-water and total degradation of proteins accomplished by hydrazinolysis. Anion exchange and size exclusion chromatography were used to separate neutral polysaccharides from oligosaccharides. Parallel studies on non-diseased brains indicated (FIG. 1) that polysaccharides with the highest molecular weight were present in much higher proportion in the diseased brain than in the normal brain. Further separation and analyses of these high molecular weight fractions by hydrolysis, reduction, peracetylation and GC-MS indicated that the highest molecular weight peak from the diseased brains contained a component comprised exclusively of glucose that accounted for 5% of the total neutral polysaccharide content. This fraction was absent in the control brains.

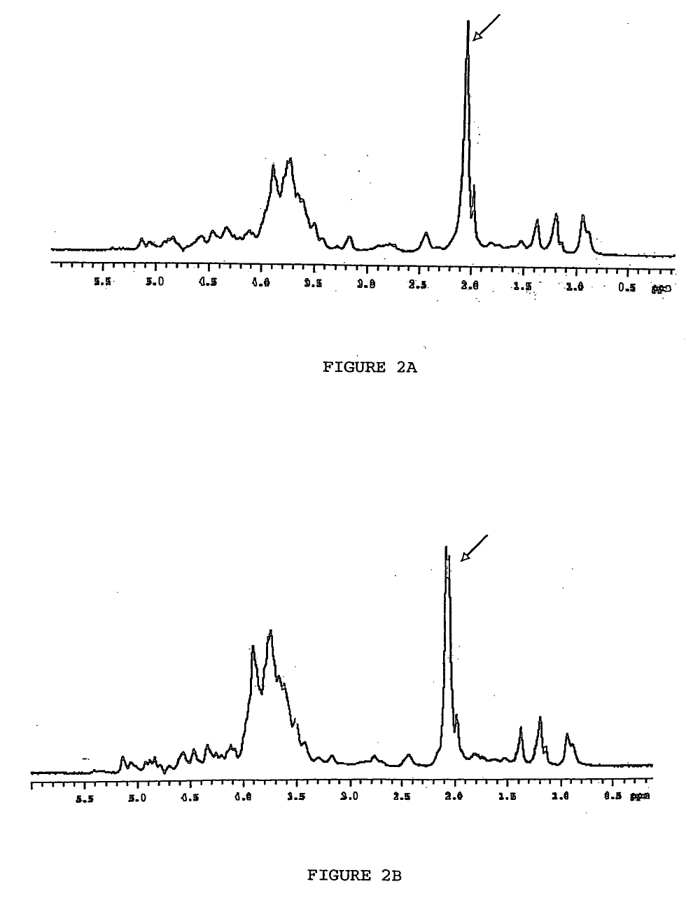

Methylation analysis (Hakamori, J. Biochem. 55: 205-208 (1964)) indicated that the glucose was 1,4-linked and NMR spectroscopy indicated that the linkage was exclusively α-linkage (δ_{1H}=5.4 ppm, J_{H-H}<4Hz). Size exclusion chromatography (HPLC) was used to determine that the molecular weight was >40,000 Daltons. This material was therefore amylose. In contrast, GC-MS analyses indicated that polysaccharides with molecular weights ranging from 40,000 to 7,000 Da. contained predominantly N-acetylglucosamine with low-level substitution by galactose and mannose and some deoxy sugars. The NMR spectra (FIG. 2) of the N-acylated material indicated that it was composed almost exclusively of N-acetyl glucosamine (N-acetyl group at 2.0 ppm and signals corresponding to only one sugar moiety between 3.0 and 4.2 ppm).

The inventors reasoned that unfunctionalized or higher molecular weight polymers of glucosamine, if present, might be insoluble because the only known polymer of glucosamine is the α-1,4-linked insoluble macromolecule chitin found in fungi and insects and in the shells of crustaceans. If this were so, the fibrous and highly insoluble nature of chitin might then feature in the well known morphology of amyloid plaque. The presence of α-1,4-linked polysaccharides such as cellulose and chitin can be specifically demonstrated by fluorescence labeling with the fluorescent dye calcofluor (Haigler et al., Science 210: 903-6 (1980)). Cytochemical studies of sections of AD tissue revealed the presence of brightly fluorescing amyloid plaques (FIGS. 3A and 3B). Wispy fibrils that might have been dislodged during the sectioning were sometimes seen outside the plaques (FIG. 3C). Fibrils were also observed in association with blood vessels (FIG. 3D). In contrast, diffusely distributed islands of granular calcofluor-stained material were present in aged control brains (FIG. 3F). The calcofluor staining supported the inference that the Alzheimer diseased brain might contain chitin.

In order to definitively confirm the presence of chitin a series of chemical, physical and physicochemical analyses and experiments were carried out. A sample of AD tissue was delipidated, treated with nucleases to remove DNA and RNA, with amylase to remove amylose, and then subjected to exhaustive enzymatic proteolysis to remove proteins. After repeated extraction with water and exhaustive dialysis the residue was placed on the top of a 15 cm column of water and allowed to slowly settle to separate the fibrils from other debris. Samples were taken at 1 cm distances along the column, recovered by centrifuging and washed several more times with water. Material from each fraction was examined by microscopy using calcofluor staining to determine which fraction contained the putative chitin fibrils. An example of the fluorescent micrograph of calcofluor fibrillar material is shown in FIG. 3E. These analyses also indicated that some fractions contained essentially only calcofluor positive material indicating that a very high level of purification had been achieved.

Unequivocal proof of the identity of the fibrils was obtained by spectroscopic and chemical analysis. Fourier transform infrared spectroscopy of single isolated fibers using an IR microscope was performed. The spectrum of a single isolated fiber was obtained (FIG. 4), matching that of a chitin standard. The IR analyses also indicated that fractions contained highly purified fibrils. Chemical analysis was also used to definitively identify the constituents of the fibrils as glucosamine. They were subjected to exhaustive acetolysis to yield peracetylated glucosamine. GC-MS analysis of the product indicated the presence of a single component the retention time and the spectrum of which (FIG. 5) was that of glucosamine peracetate.

One last important connection had to be made to associate chitin definitively as a core component of amyloid plaque. A hint to this stems from the anecdotal fact that the key histological test for amyloid plaque is based on staining with congo red, a stain that nominally does not stain tissue but is used for dying cotton. Cotton is, of course, composed of cellulose. This is a close cousin of chitin and identical in all respects both chemically and conformationally except that it contains glucose while chitin contains 2-acetamido-glucose. They both have identical flat extended structures. The definitive features of the congo red staining of amyloid plaque is that it appears red in transmitted light but yellow to yellow green when viewed between cross polarizers. Samples of commercial chitin were stained with congo red and observed under these conditions. FIG. 6A shows the typical red stain under transmitted light. FIG. 6B is a montage of images of small fibrils viewed between cross polarizers showing yellow and yellow green birefringence. Another histological stain used to characterize amyloid plaque is thioflavin-S. This also readily stained chitin. It stains only faintly with iodine, also typical of many amyloid plaques. These results taken with all of the others illustrate that chitin is a defining and critical component of amyloid plaque.

As shown herein, chitin is an important component of the highly insoluble fibrils that characterize AD pathology. These findings further provide the biochemical basis for the troubling physicochemical properties of amyloid if one were to rationalize its properties based solely on the presence of protein. Because of its physicochemical properties, chitin is ideally suited to provide a superstructure with which the many proteins that characterize the disease state and that are associated with the fibrils associate. A retrospective look at the prevailing knowledge of carbohydrate polymers may explain why chitin has not been identified until now. Chitin is totally insoluble and gives no reaction to any of the typical carbohydrate assays. On this basis, amyloid was thought to be comprised only of protein. Moreover, at the time of the assignment of the amyloid material as a protein by Friedreich and Kekule in 1859 (Virch. Arch. Path. Anal. Physiol. 16: 50-65 (1859)) the structure of chitin was not known. Although it was identified in fungi and in insects as far back as 1811 (Braconnot), its presence in higher animals was not suspected. It was some 20 years after the Friedreich and Kekule 1859 determination that it was known that chitin contained some carbohydrate-type material and acetic acid (Ledderhose, Z. Physiol. Chem. 2: 213 (1878)). Another quarter of a century would pass before Emil Fischer would show that a 2-amino-2-deoxy sugar probably with the gluco configuration was the constituent of chitin (Fischer, Leuchs. Ber. 35: 3787 (1902); Fischer, Leuchs. Ber. 36: 24 (1903)) and some 36 more years before Haworth would prove that the configuration was D-gluco-(Harworth et al., J. Chem. Soc. 271: 271 (1939)).

The biochemistry of chitin synthesis is controversial. While an important developmental role is well documented in a wide spectrum of lower organisms including yeast and bacteria (Skjak-Braek et al., In: Chitin and Chitosan (Elsevier Applied Sciences, New York, 1988)), there is evidence that chito-oligosaccharides play a, role in vertebrate development as well. Xenopus and zebrafish express two genes, xDG42 and zDG42, closely related to NodC factor of Rhizobium, during embryogenesis. Embryonic membranes from these two species were shown to synthesize chitin oligosaccharides in the absence, but not in the presence, of antibody to DG42 (Semino et al., Proc. Natl. Acad. Sci. USA 93: 4548-53 (1996)). Furthermore, microinjection of DG42 antiserum or NodC enzyme into fertilized zebrafish eggs led to severe defects in trunk and tail development (Bakkers et al., Proc. Natl. Acad. Sci. USA 94: 7982-6 (1997)). Because the transfection of DG42 into COS cells led to the synthesis of hyaluronin (Meyer and Kreil, Proc. Natl. Acad. Sci. USA 93: 4543-7 (1996)), the question arose whether DG42 is a chitin or hyaluronan synthase (Varki, Proc. Natl. Acad. Sci. USA 93: 4523-5 (1996)). Subsequently, human homologues to NodC and DG42, namely HAS1, HAS2, and HAS3, were cloned (Spicer et al., J. Biol. Chem. 272: 8957-61 (1997); Spicer and McDonald, J. Biol. Chem. 273: 1923-32 (1998)). Of these, only HAS2 and HAS3 were shown to generate substantial pericellular hyaluron coats when transfected into COS-1 cells (Itano et al. , J Biol Chem 274: 25085-92 (1999)). According to recent literature, the human homologues to NodC are considered hyaluronan synthases (Recklies et al., Biochem. J. 354: 17-24 (2001)). However, HAS1 has been shown to convert activated glucosamine to chitin (Yoshida et al., J. Biol. Chem. 275: 497-506 (2000)). In other words the product of this enzyme activity is very substrate driven. This finding has tremendous implications for the present invention since it supports the idea that a build up of glucosamine can precipitate chitin synthesis. It is also interesting to note that human synovial fluid of patients with rheumatoid or osteoarthritis contains high levels of a chitinase 3-like glycoprotein (Recklies et al., Biochem. J. 365: 119-26 (2002)), and that synovial fibroblasts from arthritic knees with HAS1, HAS2, and HAS3 messages, were shown to increase their hyaluronan synthesis in response to proinflammatory cytokines (Recklies et al., Biochem. J. 354: 17-24 (2001)). Thus, the presence of chitinase like protein suggests that not just hyaluronan but also chitin may be generated during inflammation.

The finding that chitin synthesis and fibril formation within the brain may be a precursor lesion to formation of amyloid plaques has enormous implications for the pathogenesis and treatment of AD and the aging process in general. Because diabetic tissue also contains amyloid plaque with the same histochemical features a link between these two diseases based on glucose and glucosamine metabolism is then easily established. Based on the results of this study, we suggest that inhibition of chitin synthases could have a significant impact on the morbidity and mortality of neurodegenerative disease, and possibly diabetes, where formation of chitin may have deleterious consequences on tissue function. This would represent a very clearly defined frontier for the ever increasingly important field of glycochemistry.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A method for treating a disorder in a mammal characterized by accumulation of congo red-staining plaques in a tissue of the mammal which comprises:
administering to the mammal an effective amount of a composition which inhibits polymerization of N-acetylglucosamine or derivative thereof to form chitin or conjugate thereof to treat the disorder.

2. The method of claim 1 wherein the disorder is selected from the group consisting of spongiform encepalopathies, Alzheimer's disease, hemodialysis-related amyloidosis, primary systemic amyloidosis, secondary systemic amyloidosis, familial amyloid polyneuropathy I, familial amyloid polyneuropathy III, cerebral amyloid angiopathy, Finnish hereditary systemic amyloidosis, type II diabetes, injection-localized amyloidosis, medullary thyroid carcinoma, atrial amyloidosis, non-neuropathic systemic amylodosis, and hereditary renal amyloidosis.

3. The method of claim 1 wherein the chitin or conjugate thereof is in the brain.

4. The method of claim 1 wherein the chitin or conjugate thereof is in the circulatory system.

5. The method of claim 1 or 2 wherein the chitin or conjugate thereof is in the plaques in the mammal.

6. The method of claim 1 wherein the disorder is Alzheimer's disease and the chitin or conjugate thereof is in the plaques in the brain.

7. The method of claim 1 wherein the disorder is diabetes and the chitin or conjugate thereof is in the plaques in the brain.

8. The method of claim 1 wherein the disorder is atherosclerosis and the chitin or conjugate thereof is in the plaques in the blood vessel.

9. The method of claim 1 or 2 wherein the composition comprises an inhibitor of an enzyme which produces the chitin or conjugate thereof from N-acetylglucosamine or an activated form thereof.

10. The method of claim 9 wherein composition comprises a non-natural amino acid analog of a natural amino acid to terminate formation of the chitin.

11. The method of claim 1 or 2 wherein the composition comprises an inhibitor of transcription of DNA which encodes an enzyme for producing the chitin or translation of RNA transcribed from the DNA.

12. The method of claim 1 or 2 wherein the composition comprises an inhibitor of an enzyme in a biosynthetic pathway producing the chitin from glucose.

13. The method of claim 1 or 2 wherein the composition comprises an inhibitor of a transaminase enzyme which produces an amino sugar from a keto sugar.

14. The method of claim 1 or 2 wherein the mammal is human.

15. The method of claim 1 or 2 wherein the composition comprises a chitinase which degrades the chitin.

16. The method of claim 1 or 2 wherein the composition comprises a compound selected from the group consisting of azaserine, acylurea, nikkomycin, polyoxin, polyene macrolide such as nystatin and mepartricine, and combinations thereof.

17. A method for diagnosing a disease characterized by accumulation of congo red-staining plaques in a tissue of a mammal which comprises detecting accumulated chitin or conjugate thereof in the tissue of the mammal.

18. The method of claim 17 wherein the disease is selected from the group consisting of spongiform encepalopathies, Alzheimer's disease, hemodialysis-related amyloidosis, primary systemic amyloidosis, secondary systemic amyloidosis, familial amyloid polyneuropathy I, familial amyloid polyneuropathy III, cerebral amyloid angiopathy, Finnish hereditary systemic amyloidosis, type II diabetes, injection-localized amyloidosis, medullary thyroid carcinoma, atrial amyloidosis, non-neuropathic systemic amylodosis, and hereditary renal amyloidosis.

19. The method of claim 17 or 18 wherein the mammal is living and the chitin or conjugate thereof is detected based upon instrument controlled imaging of the chitin or conjugate thereof in the tissue of the mammal.

20. The method of claim 17 or 18 wherein the tissue is from a diseased mammal.

21. The method of claim 17 or 18 wherein the mammal is human.

22. The method of claim 17 wherein the tissue is in the brain.

23. The method of claim 17 wherein the tissue is a component of the circulatory system.

24. The method of claim 17 or 18 wherein the disease is detected using a probe selected from the group consisting of a protein in a biosynthetic pathway for producing the chitin or conjugate thereof from glucose, DNA or RNA encoding a protein in a biosynthetic pathway for producing the chitin or conjugate thereof from glucose, and an antibody specific for the chitin or conjugate thereof.

25. The method of claim 17 or 18 wherein the disease is detected using a probe comprising a polypeptide fragment of a chitinase which binds the chitin or conjugate thereof without degrading the chitin or conjugate thereof.

26. A method for reducing formation of chitin or conjugate thereof in a mammal which comprises:
administering an effective amount of a composition which inhibits formation of the chitin or conjugate thereof.

27. The method of claim 26 wherein the composition comprises an antibiotic.

28. The method of claim 26 wherein the composition comprises a compound selected from the group consisting of azaserine, acylurea, nikkomycin, polyoxin, polyene macrolide such as nystatin and mepartricine, and combinations thereof.

29. The method of claim 26 wherein the composition comprises an inhibitor of a transaminase enzyme which produces an amino sugar from a keto sugar.

30. A method for reducing chitin or conjugate thereof in a mammal which comprises administering an effective amount of a chitinase to the mammal.

31. The method of claim 30 wherein the chitinase is human chitinase and the mammal is human.
